(19) United States
(12) Patent Application Publication
(10) Pub. No.: US 2025/0057814 A1
(43) Pub. Date: Feb. 20, 2025

(54) ENDODONTIC BIOFILM-ERADICATION TECHNIQUE UTILIZING A DUAL-SYSTEM INTRACANAL MEDICATION

(71) Applicant: Samer M. Ebeid, San Rafael, CA (US)

(72) Inventor: Samer M. Ebeid, San Rafael, CA (US)

(21) Appl. No.: 18/449,195

(22) Filed: Aug. 14, 2023

(51) Int. Cl. A61K 31/417; A61K 31/351; A61K 31/496; A61P 31/02

(52) U.S. Cl. CPC A61K 31/417; A61K 31/351; A61K 31/496; A61P 31/02

(57) ABSTRACT

A dual-system intracanal medicinal formulation and method are provided that target a chronic endodontic biofilm infection. The formulation include a mix of enzymes to degrade the biofilm defenses and integrity and a mix of antibiotics to eradicate the infection. After introducing the formulation into the affected root canal system and allowing the formulation to remain and eradicate the infection, the formulation is flushed and the canal system obturated.

### BACKGROUND

The root canal system is infinitely complicated with multiple channels, lateral canals, and unpredictably shaped canals. Furthermore, the Dentin structure harbors tubules which are termed dentinal tubules that can and do harbor bacteria that are resistant to elimination by conventional dental treatment protocols. Intra-canal medications have been used in endodontic treatment in the past with varying and unconvincing degrees of success. Calcium hydroxide is the most used canal medication worldwide which according to literature may or may not demonstrate strong argument for a better outcome as compared to without calcium hydroxide. In vitro studies consistently show the failure of calcium hydroxide treatment in eradication of biofilm bacteria residing in the dentinal tubules. This offers the clinician the only option in the strive for a canal hermetic seal in order to prevent such bacteria from leaking into the alveolar bone and causing disease. Ideally, the ultimate goal for endodontic treatment is the full eradication of bacteria present in the root canal system as well as the prevention of the introduction of foreign bacteria during the time treatment. This is followed by the hermetic three-dimensional seal of the canal and all its portal exits. Due to the extreme complexity of the canal system, all the dentinal tubules, the inability for instruments and irrigants to reach all parts of the canal system, as well as the treatment time for which endodontic treatment is performed only a single visit averages 60 minutes of treatment from start to finish, effective disinfection is seldom accomplished in severely infected canal system that give rise to large alveolar bone lesions. It is axiomatic to conclude that the larger the bone lesion the greater the bacterial activity within the canal system. This is demonstrated in numerous studies.

According to L Lakshmi Narayanan J Conserv Dent. 2010 October-December; 13(4): 233-239, almost 700 bacterial species can be found in the oral cavity, with any particular individual harboring 100-200 of these species. Once the root canal is infected coronally, infection progresses apically until bacterial products or bacteria themselves are able to stimulate the periapical tissues, thereby leading to apical periodontitis. Endodontic infections have a polymicrobial nature, with obligate anaerobic bacteria conspicuously dominating the microbiota in primary infections. There are various microorganisms related to intra-radicular and extraradicular infections and organisms involved in persistent infection.

### Intraradicular Infections

The endodontic pathogens that cause the primary intraradicular infections are the following. 1) Black pigmented Gram-negative anaerobic rods include the species formerly known as Bacteroides melaninogenicus. These bacteria have been reclassified into two genera: (a) saccharolytic species—Prevotella and (b) asaccharolytic species—Porphyromonas. Prevotella species detected in endodontic infections include: Prevotella intermedia; Prevotella nigrescens; Prevotella tannerae; Prevotella multissacharivorax; Prevotella baroniae and Prevotella denticola. Porphyromonas species detected in endodontic infections include: Porphyromonas endodontalis and Porphyromonas gingivalis. 2) Tannerella forsythia (previously called Bacteroides forsythus or Tannerella forsythenis) was the first periodontal pathogen to be detected in endodontic infection. 3) Dialister species are asaccharolytic obligately anaerobic Gram-negative coccobacilli which have been consistently detected in endodontic infections, including: Dialister pneumosintes and Dialister invisus. 4) Fusobacterium is also a common member of endodontic microbiota, including Fusobacterium nucleatum and Fusobacterium periodonticum. 5) Spirochetes are highly motile, spiral-shaped, Gram negative bacteria with periplasmic flagella. All oral spirochetes fall into the genus Treponema. Prevalent species are Treponema denticola; Treponema sacranskii; Treponema parvum; Treponema maltophilum and Treponema lecithinolyticum. 6) Gram positive anaerobic rods have also been found in endodontic microbiota like: Pseudoramibacter alactolyticus; Filifactor alocis; Actinomyces spp.; Propionibacterium propionicum; Olsenella spp.; Slackia exigua; Mogibacterium timidum and Eubacterium spp. 7) Gram positive cocci that are present in endodontic infection and include: Parvimonas micra (previously called Peptostreptococcus micros or Micromonas micros); Streptococcus spp. (which includes: Streptococcus anginosus; Streptococcus mitisi; Streptococcus sanguinis; Enterococcus faecalis).

Other bacterial spp. which are present in low to moderate values include: Campylobacter spp. which are Gram negative anaerobic rods (common species are: Campylobacter rectus and Campylobacter gracilis); Catonella morbic which is a saccharolytic obligate anaerobic Gram-negative rods; Veillonella parvula; Eikenella corrodens; Granulicatella adiacens; Neisseria mucosa; Centipeda periodontii; Gemella morbillorum; Capnocytophaga gingivalis; Corynebacterium matruchotii; Bifidobacterium dentium and anaerobic lactobacilli.

Apart from these, several uncultivated phylotypes which can be unrecognized but play a role in pathogenesis of apical periodontitis, such as: Dialister oral clone BSO16; Migasphaera oral clone BSO16; Solobacterium; Olsenella; Eubacterium; Cytophaga, Lachnospiraceae oral clone 55A-34; Veillonella oral clone BP 1-85; Bacteroidetes oral clone XO 83; Prevotella oral clone PUS 9.180, Eubacterium oral clone BP 1-89 and Lachnospiraceae oral clone MCE 7-60.

### Other Microorganisms in Endodontic Infections

Fungi—particularly, Candida spp. (e.g.,) Candida albicans

Archaea—These are diverse groups of prokaryotes which are distinct from bacteria. They are traditionally recognized as extremophiles but recently these microorganisms are found to thrive in non-extreme environments including human body. Methanogenic archaea have been detected in periodontal disease and chronic apical periodontitis.

The presence of viruses in the root canal has been reported only for non-inflamed vital pulps of patients infected with human immunodeficiency virus and herpes viruses where living cells are found in abundance. Among the Herpes spp., the human cytomegalovirus and Epstein-Barr virus may be implicated in the pathogenesis of apical periodontitis.

### Extraradicular Infections

Intraradicular microorganisms usually constrain themselves in the root canal due to the defense barrier. In specific circumstances, microorganisms can overcome this defense barrier and establish an extraradicular infection. This may lead to the development of acute apical abscess with purulent inflammation in periapical tissue. The extraradicular infections are dependent on or independent of an intraradicular infection. The dominant microorganisms present are anaerobic bacteria. Extraradicular microorganisms include. Actinomyces spp.; Propionibacterium propionicum; Treponema spp.; Porphyromonas endodontalis; Porphyromonas gingivalis; Treponema forsythia; Prevotella spp. And Fusobacterium nucleatum.

Bacteria Persisting Intracanal Disinfection Procedures and after Root Canal Treatment

Some microorganisms are resistant to antimicrobial treatment and can survive in the root canal after biomechanical preparation. The most common Gram-negative anaerobic rods are Fusobacterium nucleatum; Prevotella spp. And Campylobacter rectus. The most common Gram-positive bacteria are: Streptococci (Streptococcus mitis, Streptococcus gordonii, Streptococcus anginosus, Streptococcus oralis); Lactobacilli (Lactobacillus paracasei and Lactobacillus acidophilus); Staphylococci; E. faecalis; Olsenella uli; Parvimonas micra; Pseudoramibacter alactolyticus, Propionibacterium spp.; Actinomyces spp.; Bifidobacterium spp. and Eubacterium spp.

Sometimes, yeasts, commonly C. albicans, are also found in small amounts.

E. faecalis and yeast, mainly C. albicans, has been repeatedly identified as the species most recovered from root canals undergoing retreatment, in cases of failed endodontic therapy and canals with persistent infections. E. faecalis are gram positive cocci and facultative anaerobes. They are normal intestinal organisms and may inhabit the oral cavity and gingival sulcus. When this bacterium is present in small numbers, it is easily eliminated; but if it is in large numbers, it is difficult to eradicate. E. faecalis has many distinct features which make it an exceptional survivor in the root canal. These microorganisms can perform the following: live and persist in poor nutrient environment; survive in the presence of several medications (e.g., calcium hydroxide) and irrigants (e.g., sodium hypochlorite); form biofilms in medicated canals; invade and metabolize fluids within the dentinal tubules and adhere to collagen; convert into a viable but non-cultivable state; acquire antibiotic resistance; survive in extreme environments with low pH, high salinity and high temperatures; and endure prolonged periods of starvation and utilize tissue fluid that flows from the periodontal ligament.

### Biofilm

A biofilm comprises any syntrophic consortium of microorganisms in which cells stick to each other and often also to a surface. These adherent cells become embedded within a slimy extracellular matrix that is composed of extracellular polymeric substances (EPSs). The cells within the biofilm produce the EPS components, which are typically a polymeric conglomeration of extracellular polysaccharides, proteins, lipids and DNA. They are hard to eradicate because they secrete a matrix made of sugar molecules which form a kind of armor that acts as a physical and chemical barrier, preventing antibiotics from reaching their target sites within microbes.

During surface colonization bacteria cells are able to communicate using quorum sensing (QS) products such as N-acyl homoserine lactone (AHL). Once colonization has begun, the biofilm grows by a combination of cell division and recruitment. Polysaccharide matrices typically enclose bacterial biofilms. The matrix exopolysaccharides can trap QS autoinducers within the biofilm to prevent predator detection and ensure bacterial survival.

Autoinducers are signaling molecules that are produced in response to changes in cell-population density. As the density of quorum sensing bacterial cells increase so does the concentration of the autoinducer. Detection of signal molecules by bacteria acts as stimulation which leads to altered gene expression once the minimal threshold is reached. Quorum sensing is a phenomenon that allows both Gram-negative and Gram-positive bacteria to sense one another and to regulate a wide variety of physiological activities including symbiosis, virulence, motility, antibiotic production, and biofilm formation. Autoinducers come in a number of different forms depending on the species, but the effect that they have is similar in many cases. Autoinducers allow bacteria to communicate both within and between different species. This communication alters gene expression and allows bacteria to mount coordinated responses to their environments, in a manner that is comparable to behavior and signaling in higher organisms. Not surprisingly, it has been suggested that quorum sensing may have been an important evolutionary milestone that ultimately gave rise to multicellular life forms.

A biofilm may also be considered a hydrogel, which is a complex polymer that contains many times its dry weight in water. Biofilms are not just bacterial slime layers but biological systems; the bacteria organize themselves into a coordinated functional community. Biofilms can attach to a surface such as a tooth or rock and may include a single species or a diverse group of microorganisms. Subpopulations of cells within the biofilm differentiate to perform various activities for motility, matrix production, and sporulation, supporting the overall success of the biofilm. The biofilm bacteria can share nutrients and are sheltered from harmful factors in the environment, such as desiccation, antibiotics, and a host body's immune system. A biofilm usually begins to form when a free-swimming bacterium attaches to the surface.

During surface colonization bacteria cells are able to communicate using quorum sensing (QS) products such as N-acyl homoserine lactone (AHL). Once colonization has begun, the biofilm grows by a combination of cell division and recruitment. Polysaccharide matrices typically enclose bacterial biofilms. The matrix exopolysaccharides can trap QS autoinducers within the biofilm to prevent predator detection and ensure bacterial survival. In addition to the polysaccharides, these matrices may also contain material from the surrounding environment, including but not limited to minerals, soil particles, and blood components, such as erythrocytes and fibrin. The final stage of biofilm formation is known as dispersion, and is the stage in which the biofilm is established and may only change in shape and size.

### Background of Biofilm Formation

A biofilm is an assemblage of microbial cells that is irreversibly associated with a surface and is enclosed in a matrix mostly made of polysaccharide material (Donlan and Costerton, 2002). The lower layers of a biofilm contain microbes that are bound together in a polysaccharide matrix with other organic components such as eDNA, proteins, and inorganic materials. The upper layer is a loose amorphous layer extending into the surrounding medium. The fluid layer bordering the biofilm has stationary and dynamic sublayers (Chandki et al., 2011). The biofilm matrix is comprised of microbial consortia with indwelling water channels, assorted cells and extracellular polymers that are composed of glycoproteins, polysaccharides, and proteins (Christensen, 1989; Christensen and Characklis, 1990). The primary colonizers form a biofilm by auto-aggregation (attraction between same species) and co-aggregation (attraction between different species). The attached bacteria multiply and secrete an extracellular matrix, which results in a mature mixed-population biofilm (Chandki et al., 2011).

Genetic adaptation is an important mechanism of survival, which results from genetic mutations and recombination, regulation of expression of the existing genetic material, and acquisition of genetic material. The genomic plasticity or metabolic flexibility of expression within bacterial cells helps them survive rapidly changing environmental conditions and to live in diverse environmental niches (Brooks et al., 2011). Bacterial cells can colonize various parts of the human body by modifying their regulatory and metabolic activities (Yang et al., 2016). Various pathogenic bacteria possess the ability to move from the external environment to the human body by changing the nutrient uptake mechanism and the ability to resist primary and secondary immune defenses (Pickard et al., 2017). Bacterial cells can also alter their gene expression and convert from the planktonic form to the sessile form by enclosing themselves within extracellular polymeric substances (EPS) (Berlanga and Guerrero, 2016). Recent studies in the field of biofilm have focused predominantly on molecular genetics that regulate the formation of biofilms by conversion of planktonic cells to sessile forms (Davey and O'toole, 2000).

Biofilms can resist various types of antimicrobial agents (Lewis, 2001). Although more research is needed to understand the molecular mechanism behind the formation of biofilm, it is known that numerous genes change the metabolomics of bacterial cells, leading to their conversion from planktonic to sessile forms. Biofilms formed on the surface of medical devices include Gram-positive as well as Gram-negative cells. The most common Gram-positive bacteria are Enterococcus faecalis, Streptococcus pyogenes, Staphylococcus mutans, Staphylococcus epidermidis, Bacillus subtilis, and Staphylococcus aureus, whereas Gram-negative bacterial cells are Klebsiella pneumoniae, Escherichia coli, Proteus mirabilis, and Pseudomonas aeruginosa (Kwakman et al., 2006). Apart from bacteria, various groups of filamentous fungi and yeasts can form biofilms on abiotic surfaces, but these biofilms differ from those formed by bacterial cells: In yeast, the attachment is mediated by special types of proteins known as adhesion proteins (Willaert, 2018), which are usually located outside the cell wall and are subjected to epigenetic switching that results in the development of stochastic expression pattern (Verstrepen and Klis, 2006). Biofilm-derived L. pneumophila replicates more in murine macrophages than in planktonic bacteria. The biofilm is the most important determinant of survival and proliferation of bacteria in warm, humid environments.

To remove biofilms from medical devices, coatings made of acylase and α-amylase are used (Ivanova et al, 2015). The enzymes amylase, cellulase, protease, DNase, alginate, and lyase are reported to support removal of biofilms from medical devices (Stiefel et al., 2016). Therefore, enzymes can be considered natural agents for degradation of biofilm.

### Extracellular Matrix

The EPS matrix consists of exopolysaccharides, proteins and nucleic acids. This matrix encases the cells within it and facilitates communication among them through biochemical signals as well as gene exchange. The EPS matrix also traps extracellular enzymes and keeps them in close proximity to the cells. Thus, the matrix represents an external digestion system and allows for stable synergistic micro consortia of different species.

Bacteria living in a biofilm usually have significantly different properties from free-floating bacteria of the same species, as the dense and protected environment of the film allows them to cooperate and interact in various ways. One benefit of this environment is increased resistance to detergents and antibiotics, as the dense extracellular matrix and the outer layer of cells protect the interior of the community. In some cases antibiotic resistance can be increased up to 500-5,000 times (table 1). Lateral gene transfer is often facilitated within bacterial and archaeal biofilms and leads to a more stable biofilm structure. Extracellular DNA is a major structural component of many different microbial biofilms. Enzymatic degradation of extracellular DNA can weaken the biofilm structure and release microbial cells from the surface. However, biofilms are not always less susceptible to antibiotics. For instance, the biofilm form of Pseudomonas aeruginosa has no greater resistance to antimicrobials than do stationary-phase planktonic cells, although when the biofilm is compared to logarithmic-phase planktonic cells, the biofilm does have greater resistance to antimicrobials. This resistance to antibiotics in both stationary-phase cells and biofilms may be due to the presence of persister cells.

### Extracellular Polymeric Substances (EPSs)

The extracellular polymeric substances (EPSs) are natural polymers of high molecular weight secreted by microorganisms into their environment. EPSs establish the functional and structural integrity of biofilms, and are considered the fundamental component that determines the physicochemical properties of a biofilm. EPS in the matrix of biofilms provides compositional support and protection of microbial communities from harsh environments. Components of EPS can be of different classes of polysaccharides, lipids, nucleic acids, proteins, Lipopolysaccharides, and minerals.

Exopolysaccharides are the sugar-based parts of EPS. Microorganisms synthesize a wide spectrum of multifunctional polysaccharides including intracellular polysaccharides, structural polysaccharides and extracellular polysaccharides or exopolysaccharides. Exopolysaccharides generally consist of monosaccharides and some non-carbohydrate substituents (such as acetate, pyruvate, succinate, and phosphate).

As biofilm becomes established, EPS provides physical stability and resistance to mechanical removal, antimicrobials, and host immunity. Exopolysaccharides and environmental DNA (eDNA) contribute to viscoelasticity of mature biofilms so that detachment of biofilm from the substratum will be challenging even under sustained fluid shear stress or high mechanical pressure. In addition to mechanical resistance, EPS also promotes protection against antimicrobials and enhanced drug tolerance. Antimicrobials cannot diffuse through the EPS barrier, resulting in limited drug access into the deeper layers of the biofilm. Moreover, positively charged agents will bind to negatively charged EPS contributing to the antimicrobial tolerance of biofilms, and enabling inactivation or degradation of antimicrobials by enzymes present in biofilm matrix. EPS also functions as local nutrient reservoir of various biomolecules, such as fermentable polysaccharides.

Resistance to antimicrobial drugs is mediated by EPS, which renders conventional drugs ineffective. This effect has led to the drift from conventional methods of treatment to the use of other agents, such as plant secondary metabolites, antimicrobial peptides, and enzymes, as therapeutic measures (Schachtele et al., 1975, Juntarachot et al., 2020). Enzymes have been effective anti-biofilm agents, and they are environmentally friendly and easily biodegradable (Xavier et al., 2005). Enzymes can inhibit biofilms when the bacterial exopolysaccharides serve as substrate (Brisou, 1995, Sutherland, 1995). Application of suitable enzymes for degrading the structural components of the biofilm matrix will weaken it so that it can be more easily removed by mechanical processes. Since the sugar backbone of the biofilm matrix is composed mainly of carbohydrate residues, carbohydrate-based enzymes, such as amylase, might be used to hydrolyze and thereby denature the biofilm matrix (Lembre et al., 2012).

### Infectious Diseases

Biofilms have been found to be involved in a wide variety of microbial infections in the body, by one estimate 85% of all infections. Infectious processes in which biofilms have been implicated include common problems such as bacterial vaginosis, urinary tract infections, catheter infections, middle-ear infections, formation of dental plaque, [gingivitis, coating contact lenses and less common but more lethal processes such as endocarditis, infections in cystic fibrosis, and infections of permanent indwelling devices such as joint prostheses, heart valves, and intervertebral disc.

### Endodontic Biofilms

Endodontic microbiota is established to be less diverse compared to oral microbiota. Progression of infection alters the nutritional and environmental status within the root canal, making it more anaerobic with depleted nutritional levels. These changes offer a tough ecological niche for the surviving microorganisms. But complete disinfection of root canal is very difficult to achieve because of persistent microbes in anatomical complexities and apical portion of root canal. Because biofilm is the manner of bacterial growth which survives unfavorable environmental and nutritional conditions, the root canal environment will favor biofilm formation.

Endodontic bacterial biofilms can be categorized as: intracanal biofilms, extraradicular biofilms, periapical biofilms and biomaterial-centered infections.

Intracanal microbial biofilms are microbial biofilms formed on the root canal dentin of an endodontically infected tooth. Extraradicular microbial biofilms are also termed root surface biofilms which are formed on the root (cementum) surface adjacent to the root apex of endodontically infected teeth. Extraradicular biofilms are reported with asymptomatic periapical periodontitis and in chronic apical abscesses with sinus tracts. Sometimes, the extraradicular biofilm becomes calcified and gets associated with periapical inflammation and delayed periapical healing despite adequate orthograde root canal treatment. Periapical microbial biofilms are isolated biofilms found in the periapical region of endodontically infected teeth. Periapical biofilms may or may not be dependent on the root canal. These microorganisms have the ability to overcome host defense mechanisms, thrive in the inflamed periapical tissue and subsequently induce a periapical infection. Biomaterial-centered infection is caused when bacteria adhere to an artificial biomaterial surface and form biofilm structures. The presence of biomaterials in close proximity to the host immune system can increase the susceptibility to biofilm. In endodontics, biomaterial-centered biofilms form on root canal obturating materials. These biofilms can be intraradicular or extraradicular depending on whether the obturating material is within the root canal or has extruded beyond the root apex

TABLE 1
Susceptibility of planktonic and biofilm bacteria to selected antibiotics

| | | | | Concentration |
|---|---|---|---|---|
| | | | MIC or MBC | effective |
| | | | of planktonic | against biofilm |
| | | | phenotype | phenotype |
| Reference | Organism | Antibiotic | (μg/ml) | (μg/ml) |
| 215 | S. aureus NCTC | Vancomycin | 2 (MBC) | 20^{a} |
| | 8325-4 | | | |
| 26 | Pseudomonas Aeruginosa | Imipenem | 1 (MIC) | >1,024^{b} |
| | ATCC 27853 | | | |
| 26 | E. coli ATCC 25922 | Ampicillin | 2 (MIC) | 512^{b} |
| 208 | P. pseudomallei | Ceftazidime | 8 (MBC) | 800^{c} |
| 114 | Streptococcus sanguis 804 | Doxycycline | 0.063 (MBC) | 3.15^{d} |

^{a}Concentration required for 99% reduction.
^{b}Minimal biofilm eradication concentration.
^{c}Concentration required for ~99% reduction.
^{d}Concentration required for >99.9% reduction.

### Antibiotics

Aminoglycosides include the category of traditional Gram-negative antibacterial medications that inhibit protein synthesis and contain as a portion of the molecule an amino-modified glycoside (sugar). The term can also refer more generally to any organic molecule that contains amino sugar substructures. Aminoglycoside antibiotics display bactericidal activity against Gram-negative aerobes and some anaerobic bacilli where resistance has not yet arisen but generally not against Gram-positive and anaerobic Gram-negative bacteria.

Gentamicin is a bactericidal antibiotic that works by binding the 30S subunit of the bacterial ribosome, negatively impacting protein synthesis. The primary mechanism of action is generally accepted to work through ablating the ability of the ribosome to discriminate on proper transfer RNA and messenger RNA interactions. Typically, if an incorrect tRNA pairs with an mRNA codon at the aminoacyl site of the ribosome, adenosines 1492 and 1493 are excluded from the interaction and retract, signaling the ribosome to reject the aminoacylated tRNA::Elongation Factor Thermo-Unstable complex. However, when gentamicin binds at helix 44 of the 16S rRNA, it forces the adenosines to maintain the position they take when there is a correct, or cognate, match between aa-tRNA and mRNA. This leads to the acceptance of incorrect aa-tRNAs, causing the ribosome to synthesize proteins with wrong amino acids placed throughout (roughly every 1 in 500). The non-functional, mistranslated proteins misfold and aggregate, eventually leading to death of the bacterium. A secondary mechanism has been proposed based on crystal structures of gentamicin in a secondary binding site at helix 69 of the 23S rRNA, which interacts with helix 44 and proteins that recognize stop codons. At this secondary site, gentamicin is believed to preclude interactions of the ribosome with ribosome recycling factors, causing the two subunits of the ribosome to stay complexed even after translation completes. This creates a pool of inactive ribosomes that can no longer re-initiate and translate new proteins.

### Fluoroquinolones

Quinolones are chemotherapeutic bactericidal drugs. They interfere with DNA replication by preventing bacterial DNA from unwinding and duplicating. Specifically, they inhibit the ligase activity of the type II topoisomerases, DNA gyrase and topoisomerase IV, which cut DNA to introduce supercoiling, while leaving nuclease activity unaffected. With the ligase activity disrupted, these enzymes release DNA with single- and double-strand breaks that lead to cell death. The majority of quinolones in clinical use are fluoroquinolones, which have a fluorine atom attached to the central ring system, typically at the 6-position or C-7 position. Most of them are named with the -oxacin suffix. First and second-generation quinolones are largely active against Gram-negative bacteria, whereas third and fourth generation quinolones have increased activity against Gram-positive and anaerobic bacteria. Some quinolones containing aromatic substituents at their C-7 positions are highly active against eukaryotic type II topoisomerase.

It has also been proposed that quinolone antibiotics cause oxidation of guanine nucleotides in the bacterial nucleotide pool, and that this process contributes to the cytotoxicity of these agents. The incorporation of oxidized guanine nucleotides into DNA could be bactericidal. Bacterial cytotoxicity could arise from incomplete repair of closely spaced 8-oxo-2′-deoxyguanosine in the DNA resulting in double-strand breaks.

Ciprofloxacin is a broad-spectrum antibiotic of the fluoroquinolone class. It is active against some Gram-positive and many Gram-negative bacteria. It functions by inhibiting a type II topoisomerase (DNA gyrase) and topoisomerase IV, necessary to separate bacterial DNA, thereby inhibiting cell division. Bacterial DNA fragmentation will occur as a result of inhibition of the enzymes.

### Metronidazole: Nitroimidazole Antimicrobials

Metronidazole is of the nitroimidazole class. It inhibits nucleic acid synthesis by forming nitroso radicals, which disrupt the DNA of microbial cells. This function only occurs when metronidazole is partially reduced, and because this reduction usually happens only in anaerobic bacteria and protozoans, it has relatively little effect upon human cells or aerobic bacteria.

The primary target for metronidazole is the obligate anaerobe type of bacteria. Although some efficacy towards facultative anaerobes. No effect of aerobic bacteria. Mechanism of action is the interference of the Pyruvate reduction which in turn acts like a poison.

### Bromelain

Bromelain elicits an anti-inflammatory response by reducing prostaglandin E2 (PGE-2) and cyclooxygenase-2 (COX-2) synthesis. It also inhibits bacterial enterotoxin production, exaggerates the transformation of plasminogen to plasma, interacts with intestinal secretory signaling pathways, and retards the MCF-7 cell's growth-inhibitory response in the epithelium. Bromelain is used to treat osteoarthritis, dental plaque, and gingivitis, and potentiates the therapeutic effects of some antibiotics, e.g., amoxicillin and tetracycline. It is licensed as a complementary therapeutic agent for sinus and nasal swelling and seems to be an important mucolytic agent for rhinitis, rhinosinusitis, and severe rhino-nausea. Bromelain can increase the absorption of medications, including antibiotics, such as amoxicillin and tetracycline.

Bromelain in pineapple is a type of enzyme known as a protease, which breaks other proteins apart by cutting the chains of amino acids. More specifically, bromelain is a cysteine protease, meaning that it breaks apart proteins wherever they have a cysteine amino acid. Bromelain selectively prevents proinflammatory prostaglandins' biosynthesis obviously via indirect intervention. The sensitivity of bromelain has been shown to be similar to the endogenous protease plasmin. Bromelain works on fibrinogen to have drugs like plasmin products, at least in effect. Small molecular weight active peptides are used to control prostaglandin biosynthesis and to establish conditions in a stable body. Significant amounts of orally ingested bromelain have been found to be absorbed into the bloodstream unchanged, thereby increasing the proteolytic and fibrinolytic blood activity for hours. The comparison of benefits from the aspirin-like medications with bromelain, though bromelain does not induce any of the other's unwanted side effects, shows bromelain is distinct from those from nonsteroidal anti-inflammatory medicines to the prostaglandin synthetic pathway. Although aspirin inhibits cyclooxygenase, and hence, the biosynthesis of all prostaglandins, the arachidonate cascade at the thromboxane synthetase level is assumed to be further inflamed with bromelain. Circumstantial evidence indicates the synthesis of the “proinflammatory” prostaglandins inhibited with bromelain without influencing the “anti-inflammatory” prostaglandins. Therefore, bromelain helps to restore the equilibrium of the two prostaglandins, which define the condition of the healthy organisms. Though bromelain can be used in a wide range of areas, the most traditional and established use of it is as an anti-inflammatory agent inflammation is pivotal in the development of cancer during cellular transformation, angiogenesis, proliferation, metastasis, and invasion. It has been demonstrated that suppression of chronic inflammation may reduce cancer incidence and inhibit cancer progression.

Bromelain acts as an antibacterial agent by inhibiting the growth of intestinal bacteria, such as Vibrio cholera and Escherichia coli (E. coli). Bromelain stops enterotoxin production of E. coli (ETEC) bacteria and prevents diarrhea caused by E. coli Bromelain can be utilized as an anthelmintic agent against gastrointestinal nematodes like Heligmosomoides polygyrus, Trichoderma viride, and Trichurismuris. The synergistic impact of bromelain has also been observed when used concurrently with antibiotics. It is, therefore, evident that it can be used to destroy distinct intestinal pathogenic organisms. Bromelain can treat fungal infections as well. Pityriasis lichenoides chronica is a skin disorder that produces tiny, scaling, raised spots on the skin, and bromelain can effectively heal it.

Dental caries is prevented by brushing teeth more frequently, thereby lessening the duration of tooth contact with leftover food particles. Antiplaque agents in toothpaste help prevent decay as well. As reported by Harmely et al. (2011), 5% stem bromelain is beneficial in toothpaste as an antiplaque agent. Rahmadini (2013) carried out a similar research formula in sampling rough bromelain from the hump of pineapple in toothpaste and examining their mechanical resistance for 28 days.

Bromelain can enhance tissue permeability and absorption of antibiotics after being administered orally, subcutaneously, or intramuscularly. In humans, bromelain raises levels of antibiotics in urine and blood. After bromelain is administered, higher blood and tissue levels of amoxicillin and tetracycline can be observed. As a result, higher serum and tissue levels of the drug can be maintained. Thus, bromelain potentiates the efficiency of antibiotics and lessens side effects. Diseases like pyelonephritis, cutaneous Staphylococcus infection, rectal abscesses, sinusitis, cellulitis, bronchitis, pneumonia, and thrombophlebitis can be more quickly treated by using bromelain and antibiotic therapy concurrently. Bromelain vastly increases the efficacy of antibiotics in a variety of conditions.

Bromelain is considered to be a high-value enzyme in the therapeutics field as it is an effective treatment for inflammation, cancer, osteoarthritis, severe wounds, dental plaque, gingivitis, and various pathogens. As a natural and nontoxic compound, bromelain can be used as an alternative to multiple chemical ingredients and artificially manufactured medicines. Bromelain in pineapple is a type of enzyme known as a protease, which breaks other proteins apart by cutting the chains of amino acids. Bromelain selectively prevents proinflammatory prostaglandins' biosynthesis obviously via indirect intervention. The sensitivity of the protease has been shown to be similar to the endogenous protease plasmin. The arachidonate cascaded at the thromboxane synthetase level is assumed to be further inflamed with bromelains.

### Chymotrypsin

Chymotrypsin is another serine protease produced by the pancreas that hydrolyzes the peptide bonds of tryptophan, leucine, tyrosine, and phenylalanine. Two forms of chymotrypsin (1 and 2) are synthesized by the pancreas as inactive chymotrypsinogen.

Trypsin: chymotrypsin is a widely used oral proteolytic enzyme combination to hasten repair of traumatic, surgical, and orthopedic injuries. It shows high bioavailability without losing its biological activities as an anti-inflammatory, anti-edematous, fibrinolytic, antioxidant, and anti-infective agent. Chymotrypsin is synthesized in the pancreas. Its precursor is chymotrypsinogen. Trypsin activates chymotrypsinogen by cleaving peptidic bonds in positions Arg15-Ile16 and produces π-chymotrypsin.

Catalytic mechanisms of these two proteases are similar, but their substrate specificities are different. Trypsin favors basic residues like lysine and arginine; chymotrypsin favors aromatic residues like phenylalanine, tyrosine, and tryptophan.

In vivo, chymotrypsin is a proteolytic enzyme (serine protease) acting in the digestive systems of many organisms. It facilitates the cleavage of peptide bonds by a hydrolysis reaction, which despite being thermodynamically favorable, occurs extremely slowly in the absence of a catalyst. The main substrates of chymotrypsin are peptide bonds in which the amino acid N-terminal to the bond is a tryptophan, tyrosine, phenylalanine, or leucine. Like many proteases, chymotrypsin also hydrolyses amide bonds in vitro, a virtue that enabled the use of substrate analogs such as N-acetyl-L-phenylalanine p-nitrophenyl amide for enzyme assays.

Chymotrypsin cleaves peptide bonds by attacking the unreactive carbonyl group with a powerful nucleophile, the serine 195 residue located in the active site of the enzyme, which briefly becomes covalently bonded to the substrate, forming an enzyme-substrate intermediate. Along with histidine 57 and aspartic acid 102, this serine residue constitutes the catalytic triad of the active site. These findings rely on inhibition assays and the study of the kinetics of cleavage of the aforementioned substrate, exploiting the fact that the enzyme-substrate intermediate p-nitrophenolate has a yellow color, enabling measurement of its concentration by measuring light absorbance at 410 nm.

Chymotrypsin catalysis of the hydrolysis of a protein substrate (in red) is performed in two steps. First, the nucleophilicity of Ser-195 is enhanced by general-base catalysis in which the proton of the serine hydroxyl group is transferred to the imidazole moiety of His-57 during its attack on the electron-deficient carbonyl carbon of the protein-substrate main chain (k1 step). This occurs via the concerted action of the three-amino-acid residues in the catalytic triad. The buildup of negative charge on the resultant tetrahedral intermediate is stabilized in the enzyme's active site's oxyanion hole, by formation of two hydrogen bonds to adjacent main-chain amide-hydrogens.

The His-57 imidazolium moiety formed in the k1 step is a general acid catalyst for the k−1 reaction. However, evidence for similar general-acid catalysis of the k2 reaction (Tet2) has been controverted; apparently water provides a proton to the amine leaving group.

Breakdown of Tet1 (via k3) generates an acyl enzyme, which is hydrolyzed with His-57 acting as a general base (kH2O) in formation of a tetrahedral intermediate, that breaks down to regenerate the serine hydroxyl moiety, as well as the protein fragment with the newly formed carboxyl terminus.

### Trypsin

The enzymatic mechanism is similar to that of other serine proteases. These enzymes contain a catalytic triad consisting of histidine-57, aspartate-102, and serine-195. This catalytic triad was formerly called a charge relay system, implying the abstraction of protons from serine to histidine and from histidine to aspartate, but owing to evidence provided by NMR that the resultant alkoxide form of serine would have a much stronger pull on the proton than does the imidazole ring of histidine, current thinking holds instead that serine and histidine each have effectively equal share of the proton, forming short low-barrier hydrogen bonds therewith By these means, the nucleophilicity of the active site serine is increased, facilitating its attack on the amide carbon during proteolysis. The enzymatic reaction that trypsin catalyzes is thermodynamically favorable, but requires significant activation energy (it is “kinetically unfavorable”). In addition, trypsin contains an “oxyanion hole” formed by the backbone amide hydrogen atoms of Gly-193 and Ser-195, which through hydrogen bonding stabilize the negative charge which accumulates on the amide oxygen after nucleophilic attack on the planar amide carbon by the serine oxygen causes that carbon to assume a tetrahedral geometry. Such stabilization of this tetrahedral intermediate helps to reduce the energy barrier of its formation and is concomitant with a lowering of the free energy of the transition state. Preferential binding of the transition state is a key feature of enzyme chemistry.

The negative aspartate residue (Asp 189) located in the catalytic pocket (SI) of trypsin is responsible for attracting and stabilizing positively charged lysine and/or arginine, and is, thus, responsible for the specificity of the enzyme. This means that trypsin predominantly cleaves proteins at the carboxyl side (or “C-terminal side”) of the amino acids lysine and arginine except when either is bound to a C-terminal proline, although large-scale mass spectrometry data suggest cleavage occurs even with proline. Trypsin is considered an endopeptidase, i.e., the cleavage occurs within the polypeptide chain rather than at the terminal amino acids located at the ends of polypeptides.

In a tissue culture lab, trypsin is used to resuspend cells adherent to the cell culture dish wall during the process of harvesting cells. Some cell types adhere to the sides and bottom of a dish when cultivated in vitro. Trypsin is used to cleave proteins holding the cultured cells to the dish, so that the cells can be removed from the plates.

Trypsin is commonly used in biological research during proteomics experiments to digest proteins into peptides for mass spectrometry analysis, e.g. in-gel digestion. Trypsin is particularly suited for this, since it has a very well-defined specificity, as it hydrolyzes only the peptide bonds in which the carbonyl group is contributed either by an arginine or lysine residue.

### Amylase

An amylase is an enzyme that catalyzes the hydrolysis of starch into sugars. Amylase is present in the saliva of humans and some other mammals, where it begins the chemical process of digestion. Foods that contain large amounts of sugars. The pancreas and salivary gland make amylase (alpha amylase) to hydrolyze dietary starch into disaccharides and trisaccharide which are converted by other enzymes to glucose to supply the body with energy. Plants and some bacteria also produce amylase. Specific amylase proteins are designated by different Greek letters. All amylases are glycoside hydrolases and act on α-1,4-glycosidic bonds

### α-Amylase

The α-amylases are calcium metalloenzymes. By acting at random locations along the starch chain, α-amylase breaks down long-chain saccharides, ultimately yielding either maltotriose and maltose from amylose, or maltose, glucose and “limit dextrin” from amylopectin. They belong to glycoside hydrolase family 13. The α-amylase form is also found in plants, fungi (ascomycetes and basidiomycetes) and bacteria (Bacillus)

### β-Amylase

Another form of amylase, β-amylase is also synthesized by bacteria, fungi, and plants.

### γ-Amylase

γ-Amylase (alternative names: Glucan 1,4-a-glucosidase; amyloglucosidase; exo-1,4-α-glucosidase; glucoamylase; lysosomal α-glucosidase; 1,4-α-D-glucan glucohydrolase) will cleave α(1-6) glycosidic linkages, as well as the last α-1,4 glycosidic bond at the nonreducing end of amylose and amylopectin, yielding glucose. The γ-amylase has the most acidic optimum pH of all amylases because it is most active around pH 3. They belong to a variety of different GH families, such as glycoside hydrolase family 15 in fungi, glycoside hydrolase family 31 of human MGAM, and glycoside hydrolase family 97 of bacterial forms.

Oral biofilm contains amylase binding proteins, which may indicate that that amylases play a role in establishing the biofilm (Rogers et al., 2001). For example, α-amylase in human saliva binds to α-amylase-binding proteins (ABPs) that are present on bacterial surfaces. Glucose and maltose released from processed starches by salivary amylase are metabolized by oral bacteria to form the biofilm of dental plaque. This process induces oral colonization by streptococci, which leads to the formation of oral biofilm by the plaque-forming bacteria (Nikitkova et al., 2013) The extracellular protein network of AbpA-amylase-Gtf may influence the ecology of oral biofilms, likely during initial phases of colonization. Thus, AbpA-amylase-Gtf may help in co-aggregation and colonization within the oral cavity. The functional significance of amylase binding proteins in oral colonization by Streptococci is important for understanding how salivary components influence oral biofilm formation by these important dental-plaque species. Therefore, the question is raised of whether amylase assists in the formation of dental biofilm or, paradoxically, it can be used as a biofilm inhibitor (Haase et al., 2017, Wu et al., 2020). Amylase seems to be useful for removing biofilm by disintegrating the carbohydrate moiety of the biofilm matrices, but at the same time it can also induce biofilm formation. The present review explores the evidence on the role of amylase in biofilm formation at the molecular level and the mechanisms that may be used for eradicating the biofilm.

Amylase is an important group of enzymes, which are classified into α, β, γ subtypes, isoamylase, glucoamylase, and others. α and β-amylase have the potential to catalyze the hydrolysis of chitosan (Rokhati et al., 2013) and reduce its molecular weight, which makes it more soluble (Pati et al., 2020a) and thus may lead to diversified applications (Pati et al., 2020b). Since the discovery of the first amylase by Anselme Payen, in 1833 (Krikorian, 1970), many more have been found within living systems that have specific substrates (Guzmin-Maldonado et al, 1995; Gupta et al., 2003). Amylases can be found in both plant and microbial sources. Based on the mode of action, amylases can be classified into exo-amylases and endo-amylases. Exo-amylases hydrolyze substrates from the non-reducing ends, resulting in shorter end products (Gupta et al., 2003), whereas endo-amylases act on internal glycosidic linkages in a random manner within starch molecules, resulting in oligosaccharides of various lengths (Stütz and Wrodnigg, 2011). Multiple amylases present in L. pneumophila are essential for hydrolyzing polysaccharides into glucose and in helping intracellular proliferation. Amylase also helps trigger pro-inflammatory responses, which further helps prevent bacterial replication (Douglas et al., 1990. Murray et al., 1992, Souza et al., 2020).

### α-Amylases: Structural and Functional Characteristics

α-1,4-glucan-4-glucanohydrolase, EC. 3.2.1.1, which predominantly acts on starch (polysaccharide) as the major substrate, consists of two glucose polymers—amylose and amylopectin. α-amylase helps in the hydrolysis of α-1,4 and α-1,6-glycosidic linkages, which results in the formation of small glucose (monosaccharides) and maltose (disaccharide). α-amylase is essentially a metalloenzyme, which requires metals such Ca2+, for maintaining the stability of the enzyme molecule (Saboury, 2002). Sequence alignment studies have found that α-amylases possess four conserved regions that are also present within the D strands (Møller et al., 2004). The α-amylases are present widely within plants, microorganisms, and higher animals (Kandra, 2003). The end products obtained by the action of this amylase are oligosaccharides of various length of limit dextrin and configurations (Van Der Maarel et al, 2002). The end products also consist of the of branched malto-oligosaccharides possessing 6-8 glucose units that have −1,6 and −1,4, linkages, maltose, and maltotriose (Whitcomb and Lowe, 2007). These amylase enzymes can bind with substrates via catalytic groups that catalyze breakage of the glycosidic bond (Iulek et al., 2000)

### β-Amylases: Structural and Functional Characteristics

β-amylase (E.C.3.2.1.2, α-1,4-D-maltoglucan hydrolase) can hydrolyze starch to β-maltose and β-limit dextrin (Chia et al., 2004). Most of the commercial amylases are obtained from plant sources.

### Glucoamylases: Structural and Functional Characteristics

Glucoamylase (EC 3.2.1.3) successively cleaves each glycosidic starch bond from the non-reducing end to form glucose. α-glucosidase (EC 3.2.1.20) resembles glucoamylase when the α-1,4-linkages are hydrolyzed from the non-reducing ends of alpha-glucans. However, the two enzymes adopt numerous pathways of distinct anomeric arrangements to release glucose. Glucoamylase inverts the α-d-glucose release mechanism, while alpha-glucosidase follows the retention process to generate α-d-glucose (Kumar and Satyanarayana, 2009). Most glucoamylases are multidomain enzymes that consist of a catalytic domain linked by an 0-glycosylated linker region to a starch-binding domain (Sauer et al., 2000). A glucoamylase [gamA] gene encodes a eukaryotic-like glucoamylase that is responsible for the degradation of glycogen and starch in bacteria such as Legionella pneumophila

### Human Salivary and Pancreatic Amylases: Structural and Functional Characteristics

Salivary amylase is a glucose-polymer enzyme, which cleaves large starch molecules into dextrin and subsequently into smaller malto-oligosaccharides containing α-D-(1,4) linkages, iso-malto-oligosaccharides containing α-D-(1,6) linkages, the trisaccharide maltotriose, and the disaccharide maltose (Jacobsen et al., 1972). Salivary and pancreatic amylases hydrolyze starch (Bonnefond et al, 2017). Human pancreatic amylase cannot cleave the 1,6-linkages nor the terminal glucose residues (Whitcomb and Lowe, 2007). Human amylase is a calcium-containing enzyme comprised of 512 amino acids with a single chain of oligosaccharide having a molecular weight of 57.6 kDa (Whitcomb and Lowe, 2007). The protein is comprised of three domains, namely, A, B and C. of which A is the largest and is mainly barrel shaped with eight superstructures. The B domain is located between A and C and is linked with A via disulphide bonds. The C domain has a sheet-like structure that remains attached to the A domain via a simple polypeptide chain, which appears as an independent domain having no known function. The active site of the amylase is between the carboxyl end of the A and B domains that have the calcium ion and help stabilize the three-dimensional structure (Muralikrishna and Nirmala, 2005) (FIG. 3)

### Amylase-Associated Biofilm Removal

The oral cavity contains biofilms of microbial species such as C. albicans, C. glabrata, E. faecalis, S. mutans, V. dispar and F. nucleatum (Berger et al, 2018). Though saliva is rich in amylase, plaque formation has been reported to occur in the presence of the enzyme. Natural selection has dictated the mechanisms working in vivo (not often mimicked in vitro). Amylase has potent antibiofilm activity (Kalpana et al., 2012). However, in vitro studies have shown that α-amylase is a potential antibiofilm agent against biofilm-forming bacterial species such as S. aureus and P. aeruginosa (Lahiri et al., 2021a). Although α-amylase did not have much effect on the biofilm formed by S. epidermidis, it reduced biofilm formation, and it completely inhibited biofilm formation by S. aureus (Bradford, 2011). A 79% reduction in the biofilm was observed in S. aureus when challenged with enzyme for 5 minutes. Increase in the concentration of amylase from 10 mg/mL to 100 mg/mL decreased the biofilm formation from 72% to 90% and inhibited EPS by 82% (Bradford, 2011). Six strains of MRSA had a dose-dependent response to α-amylase of about 92%-97% reduction in biofilm biomass, which is evidence that α-amylase is a potent inhibitor of biofilm formation (Watters et al., 2016).

In a study by Kalpana et al (2012), the α-amylase obtained from Bacillus subtilis had antibiofilm activity against S. aureus (MRSA), P. aeruginosa, and V. cholerae (Kalpana et al., 2012). The crude enzyme also was effective against S. aureus and P. aeruginosa and degraded the EPS, with efficacy of 51.8% to 73.1%; the purified enzyme reduced biofilm formation by 43.8% to 61.7%. Stronger antibiofilm effect was found in the work of Watters et al. (2016), where human plasma (10%) was supplemented in a culture of S. aureus biofilm; α-amylase was effective against both methicillin-sensitive and methicillin-resistant organisms. The work of Molobela et al. (2010) showed the successful use of α-amylase from Bacillus amyloliquefaciens and glucoamylase from Aspergillus niger on the Gram-negative biofilm-forming bacteria Pseudomonas fluorescence. EPS was reduced by 42.5% in the presence of the enzyme in a challenge of 90 minutes. Microscopic studies were performed to assess the reduction of biofilm and the ability of the enzyme to degrade the EPS and disperse the cells, which resulted in reduction of the biofilm (Molobela et al., 2010). Amylase (glucoamylase and amyloglucosidase units) was successfully used as a hydrolytic enzyme in controlling coaggregation in dental plaque, though it did not significantly alter bacterial viability within the plaque microcosm. (Ledder et al, 2009). Enzymatic inhibition of polysaccharides has been investigated, and α-amylase was found the most efficient enzyme (Divakaran et al., 2011).

The α-amylase produced from A. oryzae inhibits the biofilm formed by S. aureus. β-amylase is an exo-acting carbohydrolase that hydrolyzes the α-1,4-glucosidic linkages of starch only from the non-reducing end of the polysaccharide. The α-amylase can act anywhere on the substrate; thus it tends to act faster than does β-amylase (Toda et al., 1993). The enzyme prevents the surface adherence and helps in the dispersal of the cells, for instance the biofilm formed by Aggregatibacter actinomycetemcomitans when treated with dispersin B and poly-β-1,6-N-acetyl-D-glucoseamine hydrolyzing enzymes (Kaplan et al., 2003a; (Kaplan et al., 2003b; Izano et al, 2008). It has often been observed that a single enzyme is not sufficient to reduce biofilm formation. Therefore, researchers often test combined treatments of biofilms with various enzymes. The combination of levan hydrolase, amylase, and dextrin hydrolase has helped remove the biofilm on inanimate objects (Hatanaka and Sugiura, 1993), and beta-glucanase, protease, and alpha amylase in combination were effective in removing industrial slime (Wiatr, 1990).

The activity of β-amylases in inhibiting the biofilm is less than that of α-amylases. The reason for this difference is that β-amylases can be an exo-acting carbohydrase, which can hydrolyze 1,4-glucosidic linkages of the starch from the non-reducing end. This action opposes the activity of the α-amylases, which can act faster at any position on the substrate (Toda et al., 1993). Although amylases have acclaimed biofilm degrading activity, few reports are available on the biofilm-inducing activity of amylase.

To recapitulate, the endodontic infection disease process starts with the bacterial invasion of the dental pulp which will result in pulpal necrosis and the certain rise of the endodontic lesion in the alveolar bone. This was demonstrated in the Kakehashi work which was done at Harvard and published in 1965. This work utilized the Gnotobiotic rat model which clearly demonstrated that bacteria is always the cause of the endodontic infected lesion. At that time the biofilm mode of bacterial disease was not known. The goal in treatment of the endodontic infection is the eradication of the biofilm infection and therefore the healing potentials of the body will restore the original bone anatomy as to prior to infection. This is the cornerstone of modern endodontics.

Many endodontic scholars which include academia as well as clinical practice agree that full eradication of the bacterial infection is not possible at this time. This leaves the goal as the reduction of the bacteria to a sub-disease level and the attempt for the hermetic seal of the endodontic system.

### BRIEF DESCRIPTION OF THE DRAWINGS

The embodiments are illustrated by way of example and not limitation in the accompanying drawings, in which like references indicate similar elements, and in which:

FIG. 1 is a flow chart illustrating an embodiment of a method for treating an intra-canal endodontic infection.

### DETAILED DESCRIPTION

Embodiments tackle the two goals, i.e., the reduction of the bacteria to a sub-disease level, and the attempt for the hermetic seal of the endodontic system. This is the ultimate goal for a successful treatment in endodontics. The challenges in achieving this goal are substantial considering that the infection is of a biofilm nature which possesses many defenses vs its planktonic counterpart. However, the presence of the biofilm also presents an opportunity, since the Exopolysaccharide matrix, which many include simple and complex sugars as well as proteins, is susceptible to enzymatic action. In embodiments, a formula includes enzymes and antibiotics, with the enzymes disrupting the matrix defenses, which in turn allows the antibiotics to effectively permeate into the bacterial cells.

Embodiments described within disclose a technique utilizing a dual-system intracanal medication that targets an endodontic biofilm chronic infection. This type of biofilm infection rapidly develops after pulpal necrosis or pulpal bacterial invasion, which ultimately results in pulpal necrosis. In the endodontic biofilm infection, the conditions are unique and are not found practically anywhere else in the body. In the endodontic case the biofilm exists in a “dead space” where the immune response is no longer available. Without blood supply the body's defenses as well as therapeutics can no longer apply. For all intents and purposes the biofilm can exist unchallenged for the life of the tooth. Tooth extraction will resolve the problem, since with amputation the source of the infection is now removed. Typically, the only other way for infection eradication in such cases is the chemo-mechanical approach to root canal system disinfection. Due to the many challenges mentioned above, endodontic treatment is not always successful in fully eradicating the biofilm infection from the root canal system.

Use of embodiments of a method and formula have resulted in strong evidence of the eradication of the biofilm infection, at best, or a significant reduction of the bacterial load—with the reduction being to levels that have not been reported before.

In an embodiment of the formula, three antibiotics are included: Gentamycin, Ciprofloxacin, and Metronidazole. The choice of these antibiotics provides bactericidal efficacy by disturbing the internal life process of the bacteria. In embodiments, this bacterial targeting follows an enzymatic action which opens up the extracellular matrix defenses, allowing the antibiotics to access the bacteria.

Thus, an embodiment of a method and formula for endodontic biofilm eradication includes a dual enzyme-antibiotic intra-canal treatment where a combination of enzymes are employed to disperse and disrupt the formation as well as the protective function of the defense barriers of the biofilm, e.g., the extra-polysaccharide matrix as well as the protein scaffolding that support it. This disruption of cohesiveness within the biofilm, i.e., the disruption of the integrity of the biofilm, facilitates effective action of the antibiotic component of the treatment. Another benefit achieved by the embodiment is the penetration of the antibiotic through the dentinal tubules—especially at the apical third of the root. Such penetration at the apical third of the root works to eradicates the extra-radicular biofilm infection; an eradication that, at this time, may be achieved only through surgical intervention methods.

In an embodiment, the formula preferably includes (+/−50%).

| | | |
|---|---|---|
| Metronidazole | 100 | mg |
| Ciprofloxacin | 30 | mg |
| Gentamycin | 50 | mg |
| Trypsin and/or Chymotrypsin | 5 | mg (in total) |
| Bromelain | 5 | mg |
| Amylase | 10 | mg |

The rational for using each individual component of this formula is as follows. Generally, the antibiotics are chosen to be effective against the collective spectrum of bacteria found in a root canal infection. For example, Metronidazole is effective against anaerobic species, whereas the Ciprofloxacin and Gentamycin are effective against facultative anaerobes and aerobic species as well. Biofilm may be considered a multispecies society of infection that is non-actively replicating and all three antibiotics are chosen because they are effective against non-actively replicating bacteria.

In addition, these antibiotics work synergistically to eliminate bacteria through two main routes: 1) by interfering with bacterial replication; and 2) by interfering with cellular enzymes and thereby preventing cellular function. In case of Ciprofloxacin, replication is interfered with by interfering with the DNA replication process in bacterial cells. Specifically, Ciprofloxacin inhibits the action of two bacterial enzymes called DNA gyrase and topoisomerase IV, which are essential for the bacteria to replicate and divide. Gentamycin interferes with cellular function. Gentamycin is an aminoglycoside antibiotic that works by binding to the bacterial ribosome, which is the site of protein synthesis in the bacterial cell. Specifically, Gentamicin binds to a specific region of the bacterial ribosome called the 30S subunit, which prevents the ribosome from reading and translating the genetic code needed to produce essential proteins in the bacterial cell. This interference with protein synthesis leads to the accumulation of non-functional proteins and eventually, the death of the bacterial cell. With Metronidazole, the exact mechanism by which metronidazole works is not fully understood, but it is believed to be taken up by the cells and then converted into an active form that interacts with DNA to cause strand breakage and ultimately, cell death.

The enzymes are chosen to dismantle the protective defenses of the biofilm, which, as discussed, is composed primarily of a saccharide matrix in protein. It is thought that Amylase cleaves the saccharide chain and that one or both of Bromelain and the Trypsin:Chymotrypsin combination (or Trypsin or Chymotrypsin separately) dismantle the proteins by cleaving multiple amino acid bonds sites.

In an embodiment of a method of use of the formula, the individual components are kept separate until time of mixing. They are then mixed with a liquid saline solution with a pH of 7.5+/−0.5, to hydrolyze the acidic antibiotics and makes them bioactive. In an embodiment, saline may be added and mixed to obtain operator-chosen consistency determined to be suitable for the delivery system. The consistency chosen may be that of a paste easily introduced by way of a lentulo spiral. In other embodiments, other pharmaceutically acceptable carriers may be used so long as they are effective in introducing the formula into the canal. The mixture is introduced in the root canal system after initial disinfection by way of lentulo spiral and left in the canal system for 4 weeks with a provisional restoration. The intent is to fill the canal as completely as possible. A complete filling of the canal, however, is not absolutely required for success. In an embodiment, a fill of at least 75% is a lower limit. In such an embodiment in which the fill is 75%, the ratio, by weight, of the ingredients is maintained. After four (4) weeks the mixture is then flushed out and the canal system is obturated with gutta-percha. In addition, it has been found that flushing after only two (2) weeks has produced satisfactory results.

In the embodiment, the use of a lentulo spiral is exemplary and preferred, however, any means or device for carrying the formula into the canal may be used. Similarly, the use of gutta-percha is exemplary and preferred, however any means for sealing obturating the canal system may be employed.

Again, the purpose of embodiments of the formula and method is to treat an endodontic infection (root canal infection) where the infection develops into a chronic condition resulting in a progressive alveolar (jawbone) lesion where bone is absorbed and replaced with infected and inflamed tissue. The challenges for such an infection are two fold. The first challenge arises from the fact that most chronic bacterial infections are a direct result of a persistent biofilm infection. This means it is a multi-organism infection, extremely difficult to treat due to bacterial defense mechanisms acquired by biofilm formation. The second challenge arises from the fact that the endodontic infection resides in dead space within the canal system. Being within this dead space, which results from pulpal necrosis, prevents the immune response from reaching and being effective against the infection.

After treatment with the formula and the flushing, the dental pulp of the affected tooth may be completely inert/dead/removed. The dental pulp is not necessary to sustain the tooth after full tooth development. The tooth can survive without the dental pulp indefinitely. Thus, if the root canal system left behind after pulpal necrosis is infection-free, the tooth is sealed in order to prevent system infection from returning.

This formula was used on patients with their informed consent. Remarkable results were obtained in which the lesion was completely resolved in as little as six months. Such results mimic the results of tooth extraction. Since tooth extraction eliminates the biofilm infection completely, the fact that the results from the use of the embodiment of the formula and method provide a similar outcome demonstrates the effectiveness of the formula and method. This high level of healing outcome has never been shown with any other intracanal medication.

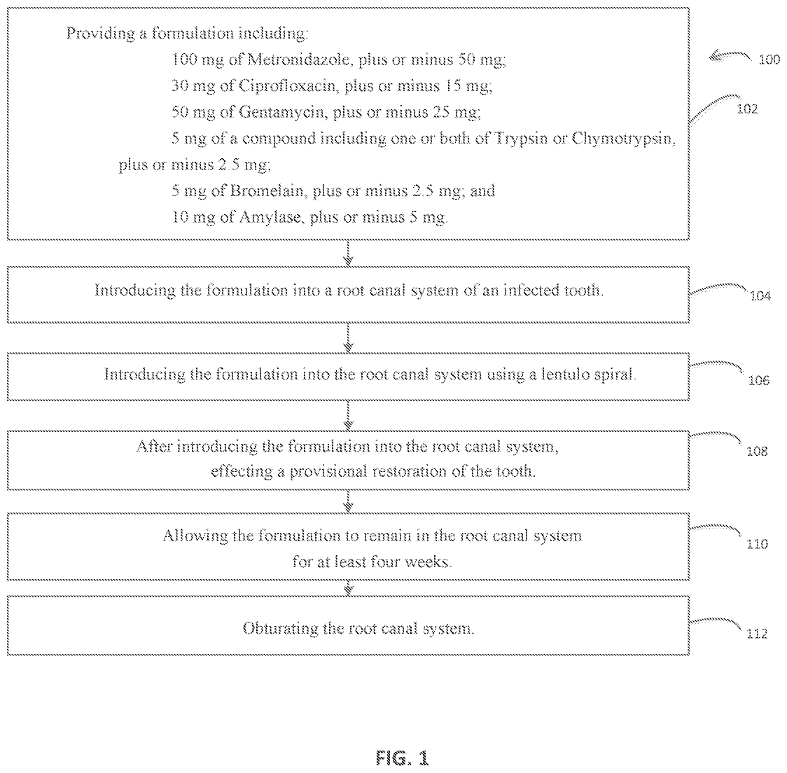

FIG. 1 is a flow chart illustrating an embodiment of a method 100 for treating an intra-canal endodontic infection. In method 100, in step 102, a medicinal formulation is provided, the formulation including: 100 mg of Metronidazole, plus or minus 50 mg, 30 mg of Ciprofloxacin, plus or minus 15 mg; 50 mg of Gentamycin, plus or minus 25 mg; 5 mg of a compound including one or both of Trypsin or Chymotrypsin, plus or minus 2.5 mg; 5 mg of Bromelain, plus or minus 2.5 mg; and 10 mg of Amylase, plus or minus 5 mg. In step 104, the formulation is introduced into a root canal system of an infected tooth. In an optional step 106, the formulation is introduced into the root canal system using a lentulo spiral. In an optional step 108, after introducing the formulation into the root canal system, a provisional restoration of the tooth is effected. In an optional step 110, the formulation is allowed to remain in the root canal system for at least four weeks. In a step 112, the root canal system is obturated.

### EXAMPLES

Cases were chosen that showed a lesion so advanced that a negative outcome was anticipated if treated with conventional treatment or with uses of calcium hydroxide. These included and were not limited to large endodontic lesions which measured 6 mm or greater. A large lesion is defined as 6 mm or greater as measured on the cone beam CT scan. Further symptoms included: significant bone loss at bony plates, maxillary sinus invasion, persistent symptoms, secondary apical-marginal defects which resulted in periodontal defects (which destroy the biologic attachment of the tooth), internal and external resorption cases, and apical resorption of roots which involve large lesions. A pre-op cone-beam computed tomography (CBCT) was taken on every case. According to endodontic research, endodontic lesions may take four years or longer for radiographic resolution. A 2D radiograph is the how the majority of publications measure the healing of such lesions. However, a 2D radiograph is often misleading and less representative of the actual healing. Since a CBCT is the most accurate display of bone healing and new bone growth, it was used to evaluate the healing in each exemplary case. In each case, most of the healing occurred in six months. Where a longer time was needed, the lesion was extremely large and needed more time to heal simply due the rate at which new bone grows. Since a conventional tooth extraction fully eliminates the endodontic disease, any new endodontic treatment should preferably also eliminate the endodontic disease to the same level. In over 93% of the cases that returned for follow up, the use of the embodiments of the formula and method showed complete bone healing within twelve months, which exceeds the exceeds the healing criteria of the American Association of Endodontists.

### Example Case No. 1

In Case No 1, a large lesion involved two teeth with complete buccal bony plate destruction. The embodiment of the formula included: Metronidazole (100 mg); Ciprofloxacin (30 mg); Gentamycin (50 mg), Trypsin and Chymotrypsin (5 mg in total in a ratio of approximately 1.1)), Bromelain (5 mg) and Amylase (10 mg). Saline was added and mixed to obtain operator-chosen consistency that was determined by the consistency suitable for the delivery system. The consistency was that of a paste easily introduced by way of a lentulo spiral. Use of an embodiment of the intra-canal formula treatment showed complete bone healing and buccal plate regeneration at the six month post-op follow-up. Tooth #5 initially had a large endodontic lesion with involvement of both buccal and palatal bony plates. The lesion also involved the distal tooth. The endodontic treatment included four weeks of intracanal medication using the intra-canal formula. A six month post-op radiograph showed complete healing with full bone resolution. A CT scan showed full bone healing at the six month post-op. In addition, the CT scan showed full resolution of bony plate defects.

### Example Case No. 2

In Case No. 2 a large endodontic lesion caused significant bone loss including a buccal bony plate defect. Two draining sinus tracts were also present and traced in a pre-op radiograph. The radiograph revealed a periodontal defect probing to the apex of the tooth buccal. The endodontic treatment included four weeks of intra canal medication using the intra-canal formula and delivery system of Example Case No. 1. A six month post-op evaluation included a periapical radiograph as well as a post-op cone beam CT scan. Complete bone resolution was seen on the post-op scan. The 3D CT scan rendition showed buccal bone growth back to the original anatomical position confirming complete healing.

### Example Case No. 3

Case No. 3 included a side-by-side comparison of the intra canal formula and calcium hydroxide intra canal medication. Preop radiographs showing lesions. Teeth 7 and 8 presented with a large lesion. Tooth 10 presented a smaller lesion. A 3D scan shows buccal plate destruction for teeth 7 and 8 but not tooth 10. Teeth 7 and 8 were treated with the intra-canal formula and delivery system of Example Case No. 1 and tooth 10 was treated with calcium hydroxide. All teeth were treated by the same operator using the same techniques of canal disinfection, four weeks of intra-canal medication, and all teeth were treated at the same time. The post-op follow-up was done after 12 months post obturation. The post-op showed healing of the large lesion on teeth 7 and 8 (the intra-canal formula treated lesion), and non-healing of the smaller lesion 10 (the calcium hydroxide-treated lesion). Furthermore, the calcium hydroxide-treated tooth showed no change in the size of lesion. A radiograph taken twelve months post op showed resolution of the lesion on the side treated with the intra-canal formula and full regeneration of the buccal bony plate. The same radiograph showed no change in healing of calcium hydroxide side.

The previous description is provided to enable any person skilled in the art to practice the various aspects described herein. While the foregoing disclosure has been described in some detail for purposes of clarity and understanding, it will be clear to one skilled in the art from a reading of this disclosure that various changes in form and detail can be made without departing from the true scope of the invention. It is to be understood that the invention is not limited to any of the specifically recited methodologies, reagents, biological materials or instrumentation that are recited herein, where similar or equivalent methodologies, reagents, biological materials or instrumentation can be substituted and used in the construction and practice of the invention, and remain within the scope of the invention. It is understood that the description and terminology used in the present disclosure is for the purpose of describing particular embodiments of the invention only, and is not intended that the invention be limited solely to the embodiments described herein.

As used in this specification and the appended claims, singular forms such as “a”, “an” and “the” include plural referents unless the content clearly dictates otherwise. All industry and technical terms used herein have the same meaning as commonly understood by one of ordinary skill in the art or industry to which the invention pertains, unless defined otherwise. A phrase such as an “aspect” does not imply that such aspect is essential to the subject technology or that such aspect applies to all configurations of the subject technology. A disclosure relating to an aspect may apply to all configurations, or one or more configurations. A phrase such as an aspect may refer to one or more aspects and vice versa. A phrase such as a “configuration” does not imply that such configuration is essential to the subject technology or that such configuration applies to all configurations of the subject technology. A disclosure relating to a configuration may apply to all configurations, or one or more configurations. A phrase such as a configuration may refer to one or more configurations and vice versa. Various modifications to these aspects will be readily apparent to those skilled in the art, and the generic principles defined herein may be applied to other aspects. Thus, the claims are not intended to be limited to the aspects shown herein, but are to be accorded the full scope consistent with the language claims, wherein reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the subject disclosure. All structural and functional equivalents to the elements of the various aspects described throughout this disclosure that are known or later come to be known to those of ordinary skill in the art are expressly incorporated herein by reference and are intended to be encompassed by the claims.

All publications, patents, patent applications, and/or other documents cited in this application are incorporated by reference in their entirety for all purposes to the same extent as if each individual publication, patent, patent application, and/or other document were individually indicated to be incorporated by reference for all purposes.

What is claimed is:

1. A pharmaceutical composition comprising:
Metronidazole,
Ciprofloxacin;
Gentamycin;
one or both of: Trypsin or Chymotrypsin;
Bromelain; and
Amylase.

2. The pharmaceutical composition of claim 1, wherein a dose of the composition includes:
100 mg of Metronidazole, plus or minus 50 mg;
30 mg of Ciprofloxacin, plus or minus 15 mg;
50 mg of Gentamycin, plus or minus 25 mg;
5 mg of a compound including one or both of Trypsin or Chymotrypsin, plus or minus 2.5 mg;
5 mg of Bromelain, plus or minus 2.5 mg; and
10 mg of Amylase, plus or minus 5 mg.

3. The pharmaceutical composition of claim 1, wherein the composition, when in solid form, includes by weight:
50% Metronidazole, plus or minus 25%;
15% of Ciprofloxacin, plus or minus 7.5%;
25% of Gentamycin, plus or minus 12.5%;
2.5% of a compound including one or both of Trypsin or Chymotrypsin, plus or minus 1.25%;
2.5% of Bromelain, plus or minus 1.25%; and
5% of Amylase, plus or minus 2.5%.

4. The pharmaceutical composition of claim 1, further comprising a pharmaceutically acceptable carrier.

5. The pharmaceutical composition of claim 4, wherein the pharmaceutically acceptable carrier is a saline solution.

6. The pharmaceutical composition of claim 5, wherein the saline solution is at a pH of 7.5, +/−0.5.

7. A method for treating an intra-canal endodontic infection comprising:
introducing a formulation into a root canal system of an infected tooth of a subject, the formulation including:
Metronidazole;
Ciprofloxacin;
Gentamycin;
one or both of: Trypsin or Chymotrypsin;
Bromelain; and
Amylase.

8. The method of claim 7, wherein the formulation includes:
100 mg of Metronidazole, plus or minus 50 mg;
30 mg of Ciprofloxacin, plus or minus 15 mg;
50 mg of Gentamycin, plus or minus 25 mg;
5 mg of a compound including one or both of Trypsin or Chymotrypsin, plus or minus 2.5 mg;
5 mg of Bromelain, plus or minus 2.5 mg; and
10 mg of Amylase, plus or minus 5 mg.

9. The method of claim 7, wherein the formulation, when in solid form, includes by weight:
50% Metronidazole, plus or minus 25%;
15% of Ciprofloxacin, plus or minus 7.5%;
25% of Gentamycin, plus or minus 12.5%;
2.5% of a compound including one or both of Trypsin or Chymotrypsin, plus or minus 1.25%;
2.5% of Bromelain, plus or minus 1.25%; and
5% of Amylase, plus or minus 2.5%.

10. The method of claim 7, wherein the formulation further comprises a pharmaceutically acceptable carrier.

11. The method of claim 10, wherein the pharmaceutically acceptable carrier is a saline solution.

12. The method of claim 11, wherein the saline solution is at a pH of 7.5, +/−0.5.

13. The method of claim 7, wherein introducing a formulation into a root canal system of an infected tooth of a subject includes:
introducing the formulation into the root canal system using a lentulo spiral.

14. The method of claim 7, further comprising:
allowing the formulation to remain in the root canal system for at least two weeks.

15. The method of claim 7, further comprising:
allowing the formulation to remain in the root canal system for at least four weeks.

16. The method of claim 7, further comprising:
after introducing the formulation into the root canal system, effecting a provisional restoration of the tooth.

17. The method of claim 7, wherein introducing the formulation into the root canal system includes:
introducing the formulation into the root canal system until the root canal is at least 75 percent filled with the formulation.

18. The method of claim 7, further comprising:
flushing the formulation from the root canal system; and
obturating the root canal system.

19. The method of claim 18, wherein the root canal system is obturated with gutta-percha.

20. A pharmaceutical composition comprising:
Metronidazole;
Ciprofloxacin;
Gentamycin;
one or both of: Trypsin or Chymotrypsin;
Bromelain; and
Amylase, in percentages as determined by weight and equal to a compound including:
100 mg of Metronidazole, plus or minus 50 mg;
30 mg of Ciprofloxacin, plus or minus 15 mg;
50 mg of Gentamycin, plus or minus 25 mg;
5 mg of a compound including one or both of Trypsin or Chymotrypsin, plus or minus 2.5 mg;
5 mg of Bromelain, plus or minus 2.5 mg; and
10 mg of Amylase, plus or minus 5 mg.
